(19) United States
(12) Patent Application Publication
(10) Pub. No.: US 2026/0279588 A1
(43) Pub. Date: Sep. 17, 2026

(54) MACHINE LEARNING PREDICTION MODELS OF OUTCOME FROM CROSS-SECTIONAL VASCULAR IMAGES

(71) Applicants: MEDSTAR HEALTH, INC., Columbia, MD (US); NATIONAL LABORATORY FOR SCIENTIFIC COMPUTING, Rio de Janeiro (BR); Instituto Pladema, Universidad del Centro, Consejo Nacional de Investigaciones Cientificas y, Buenos Aires (AR); FLOUIT, INC, Buenos Aires (AR); INSELSPITAL, UNIVERSITY HOSPITAL BERN, Bern (CH)

(72) Inventors: Hector GARCIA-GARCIA, Columbia, MD (US); Pablo Javier BLANCO, Rio de Janeiro (BR); Carlos Alberto BULANT, Buenos Aires (AR); Lorenz RÄBER, Bern (CH); Gonzalo Damián ARES, Mar del Plata (AR)

(21) Appl. No.: 19/472,137

(22) PCT Filed: Apr. 3, 2024
(86) PCT No.: PCT/US2024/022728

§ 371 Date: Oct. 3, 2025

Related U.S. Application Data
(60) Provisional application No. 63/493,785, filed on Apr. 3, 2023.
(60) Provisional application No. 63/591,518, filed on Oct. 19, 2023.

(51) Int. Cl. G16H 50/30; G06T 7/00; G06V 10/25; G06V 10/764; G06V 10/766; G06V 10/82; G16H 50/20

(52) U.S. Cl. CPC G16H 50/30; G06T 7/0012; G06V 10/25; G06V 10/764; G06V 10/766; G06V 10/82; G16H 50/20; G06T 2207/10132; G06T 2207/20081; G06T 2207/20084; G06T 2207/30101; G06V 2201/03

(57) ABSTRACT

Systems and methods of predicting progression or regression of arterial plaque are discussed. One example method includes accessing a set of segmented intravascular ultrasound (IVUS) image frames of a blood vessel of a patient. The method also includes defining a set of regions of interest (ROIs) of the set of segmented IVUS image frames. Additionally, the method includes determining a set of features for a ROI of the set of ROIs. The method further includes providing the set of features to a trained plaque prediction algorithm. The method additionally includes receiving a prediction for the ROI from the trained plaque prediction algorithm, wherein the prediction indicates one of plaque progression, plaque regression, or stationary plaque.

### RELATED APPLICATIONS

This application claims priority to U.S. Provisional Patent No. 63/591,518, filed Oct. 19, 2023 and entitled “Machine Learning Prediction Models Of Coronary Plaque Progression After One-Year Of High-Intensity Rosuvastatin Therapy From Intravascular Ultrasound Images” and No. 63/493,785, filed Apr. 3, 2023 and entitled “Machine Learning Prediction Models Of Coronary Plaque Progression After One-Year Of High-Intensity Rosuvastatin Therapy From Intravascular Ultrasound Images,” both of which are hereby incorporated by reference in their entireties.

### TECHNICAL FIELD

This invention relates to medical imaging, and more particularly, to predicting coronary plaque progression after therapy from intravascular ultrasound imaging.

### BACKGROUND

Coronary artery disease (CAD) is defined by the existence of atherosclerotic plaque on the arterial wall, which can cause blood flow impairment, or plaque rupture, and ultimately lead to myocardial ischemia. Intravascular ultrasound (IVUS) imaging can provide a detailed characterization of lumen and vessel features, and thus plaque burden, in coronary vessels. Prediction of the regions in a vascular segment where plaque burden can either increase (progression) or decrease (regression) following a certain therapy has remained an elusive major milestone in cardiology. Studies have shown an association between plaque burden regression and high intensity rosuvastatin therapy over 13 months. Nevertheless, it has not been possible to predict if a patient would respond in a favorable/adverse fashion to such a treatment.

### SUMMARY OF THE INVENTION

In one implementation, a machine-readable medium having machine executable instructions that cause a processor core to execute operations is provided. The operations include accessing a set of segmented cross-sectional image frames of a blood vessel of a patient. The operations also include defining a set of regions of interest (ROIs) of the set of segmented cross-sectional image frames. The operations additionally include determining a set of features for a ROI of the set of ROIs. The operations further include providing the set of features to a trained outcome prediction algorithm. Additionally, the operations include receiving a prediction for the ROI from the trained outcome prediction algorithm, wherein the prediction indicates one of a patient-level outcome, plaque progression, plaque regression, or stationary plaque.

In another implementation, a system is provided. The system includes a memory for storing machine-readable instructions and a processor core for accessing the machine-readable instructions and executing the machine-readable instructions as operations. The operations include accessing a set of segmented cross-sectional image frames of a blood vessel of a patient. The operations also include defining a set of regions of interest (ROIs) of the set of segmented cross-sectional image frames. The operations additionally include determining a set of features for a ROI of the set of ROIs. The operations further include providing the set of features to a trained outcome prediction algorithm. Additionally, the operations include receiving a prediction for the ROI from the trained outcome prediction algorithm, wherein the prediction indicates one of a patient-level outcome, plaque progression, plaque regression, or stationary plaque.

In a further implementation, a method is provided. The method includes accessing a set of segmented cross-sectional image frames of a blood vessel of a patient. The method also includes defining a set of regions of interest (ROIs) of the set of segmented cross-sectional image frames. The method additionally includes determining a set of features for a ROI of the set of ROIs. The method further includes providing the set of features to a trained outcome prediction algorithm. Additionally, the method includes receiving a prediction for the ROI from the trained outcome prediction algorithm, wherein the prediction indicates one of a patient-level outcome, plaque progression, plaque regression, or stationary plaque.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 illustrates one example of a system for predicting whether arterial plaque will progress or regress in one or more regions of interest of a blood vessel.

FIG. 2 illustrates a baseline and follow-up pullback pair of intravascular ultrasound (IVUS) images.

FIG. 3 illustrates a method of training and testing the prototype algorithm that was performed at each iteration.

FIG. 4 illustrates a flow diagram showing a flow diagram of performing one regression/classification experiment in connection with the prototype.

FIG. 5 illustrates a table showing basic statistics for the difference in percentage atheroma volume between follow-up and baseline IVUS pullbacks, considering the different criteria to define the region of interest (ROI) partitions.

FIG. 6 illustrates the statistics of the prediction metrics for the two best performing ROI definitions as a result of training and testing with the same repeated stratified k-fold cross-validation procedure.

FIG. 7 shows the selection count and total mean absolute Shapley values times the selection ratio values for multiple clinical features.

FIG. 8 shows mean absolute Shapley values of the five most influential features for the two best performing ROI definitions.

FIG. 9 illustrates a flowchart of an example method for training and validating an outcome prediction algorithm to predict an outcome in response to a potential treatment based on regions of interest of a set of cross-sectional vascular images.

FIG. 10 illustrates a flowchart of an example method for employing an outcome prediction algorithm to predict whether an outcome in response to a potential treatment based on regions of interest of a set of cross-sectional vascular images.

FIG. 11 is a schematic block diagram illustrating an exemplary system of hardware components capable of implementing examples of the systems and methods disclosed herein.

### DETAILED DESCRIPTION

In the context of the present disclosure, the singular forms “a,” “an” and “the” can also include the plural forms, unless the context clearly indicates otherwise. The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “substantially identical” or “substantially equal” refers to articles or metrics that are identical other than measurement error.

As used herein, an “intravascular image” is an image that includes an interior of a blood vessel. Such images can be produced, for example, via intravascular ultrasound (IVUS) or optical coherence tomography (OCT).

Various examples discussed herein are employable with a variety of imaging modalities, both intravascular and non-intravascular, that obtain cross-sectional views of a blood vessel or sufficient data from which cross-sectional views of the blood vessel can be obtained. However, for ease of illustration, some examples are discussed specifically in connection with IVUS images and pullbacks.

The systems and methods described herein provide a framework that processes lumen and vessel cross-sectional contours and extracts geometric descriptors from baseline and follow-up cross-sectional views of blood vessels (e.g., IVUS pullbacks, etc.). The systems and methods described herein also provide a machine learning model trained and validated on baseline/follow-up cross-sectional views of blood vessels (e.g., IVUS datasets, etc.) that predict an outcome in connection with the blood vessel using baseline information (e.g., geometric features, anatomic features, hemodynamic features, clinical data, etc.).

In some examples, the predicted outcome includes future precent of atheroma volume changes (e.g., plaque progression or regression) in vascular segments (e.g., regions of interest (ROIs)) based on features associated with those ROIs. In the same or other examples, the predicted outcome includes the impact on a vascular segment (e.g., ROI) based on features associated with one or more other ROIs.

Additionally, in the same or other examples, the predicted outcome includes a patient-level outcome such as whether or not the patient will have an adverse cardiovascular event (e.g., a probability of occurrence of the patient-level outcome, whether the patient-level outcome is predicted to have at least a threshold probability of occurrence, etc., Because plaque progression correlates with such outcomes, some examples predict patient-level outcomes based on predictions of plaque progression/regression. Additionally or alternatively, training and validation on datasets having known associations (e.g., in connection with follow-up imaging, etc.) with the presence or absence of patient-level outcomes provide an additional basis for identifying features (e.g., geometric, anatomic, hemodynamic, clinical, etc.) associated with the presence or absence of those patient-level outcomes.

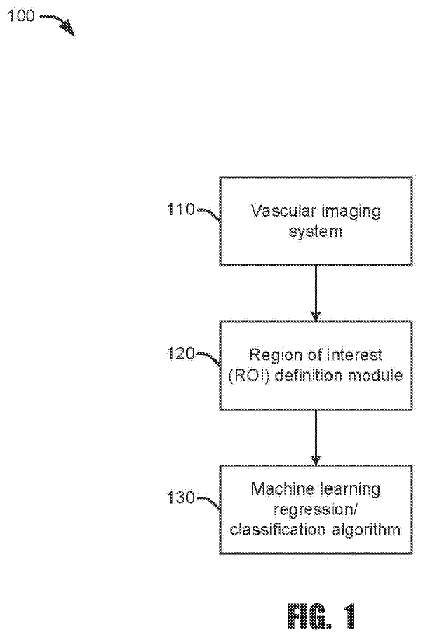

FIG. 1 illustrates one example of a system 100 for predicting whether arterial plaque will progress or regress in one or more regions of interest of a blood vessel (e.g., coronary artery). A vascular imaging device 110 (e.g., intravascular or non-intravascular) is configured to capture cross-sectional vascular images (e.g., or sufficient data from which cross-sectional vascular images can be generated, such as off-axis data, etc.). For example, the vascular imaging device 110 can be an intravascular device can include an ultrasound probe mounted on a tip of a catheter that captures images while positioned within the blood vessel or an OCT imager. In the ultrasound implementation, a series of image frames can be captured at regular intervals while the catheter tip is slowly translated through the vessel, whereas the OCT device naturally provides a series of two-dimensional slices representing a three-dimensional volume (e.g., an IVUS pullback). The captured image frames are segmented (e.g., manually or automatically) to create a segmented lumen and segmented plaque on each captured image. The segmented captured images are accessed by a region of interest (ROI) definition module 120 that defines a set of ROIs based on one or more criteria (e.g., fixed-length ROIs; ROIs based on lumen and/or external elastic membrane (EEM) measurements such as area or other features; ROIs based on a plaque burden of the image frame, such as categories based on plaque burden value or plaque burden signal and/or postprocessing signals such as a plaque burden derivative (e.g., defining ROIs as consecutive regions of positive or negative values of the first derivative of plaque burden, etc.); ROIs defined based on other criteria used in connection with the prototype; etc.).

For each ROI, a set of features (e.g., one or more of, or one or more features derived from: clinical features of the patient, geometric features, condensed features of the ROI, etc.) are determined and provided to a trained outcome prediction algorithm, for example a machine learning algorithm 130 trained for regression or classification of the set of features to predict one or more of whether the plaque in the ROI (and/or one or more other ROIs) will progress, regress, or remain stationary (and/or change by a predicted amount or percent), whether a patient associated with the captured image frames will experience a given outcome (e.g., cardiovascular event, etc.), etc. The predicted outcome is received from the machine learning algorithm 130 and can be output (e.g., via a display, etc.) for access by a clinician, etc.

Systems and methods described herein process lumen and vessel cross-sectional contours and extract geometric descriptors from baseline and follow-up cross-sectional vascular images (e.g., IVUS pullbacks). A machine learning model trained and validated based on baseline/follow-up cross-sectional vascular image datasets (and/or additional data, such as clinical data, anatomic features, hemodynamic features, etc.) is employed by systems and methods to predict an outcome associated with baseline cross-sectional vascular images using baseline information (e.g., geometric features, anatomic features, hemodynamic features, clinical data, etc.), such as future percent of atheroma volume changes in coronary vascular segments or patient-level outcomes.

A prototype employing XGBoost (a scalable, distributed gradient-boosted decision tree (GBDT) machine learning algorithm) regression models was developed based on a post hoc analysis, revisiting a prior study (the Integrated Biomarkers and Imaging Study-4 (IBIS-4) clinical trial, which showed that high-intensity rosuvastatin therapy over 13 months was associated with regression of coronary atherosclerosis in 146 non-infarct-related arteries from 82 patients). A subset of the IBIS-4 dataset (140 IVUS pullbacks from 81 patients) was employed, for which expert delineation of lumen and vessel contours were available at baseline (BL) and 13-month follow-up (FU). Contour data from BL and FU pullbacks were co-registered and then processed to extract several frame-wise features (e.g., areas, plaque burden, eccentricity, etc.). Each pullback was divided into regions of interest (ROIs), following different criteria. Frame-wise features were condensed into region-wise markers using tools from statistics, signal processing, and information theory. Finally, a stratified 5-fold cross-validation strategy (20 repetitions) was used to train/validate XGBoost regression models (in various examples other strategies and/or numbers of repetitions are used for training and/or validation). A feature selection method before the model training was also applied.

Geometric features extracted from lumen and vessel contours from IVUS images and patient data were used to train XGBoost models. When such models were trained/validated on ROI defined by the difference between follow-up and baseline plaque burden, the average Accuracy and Mathews Correlation Coefficient were 0.70 and 0.41 respectively. Using a ROI partition criterion based only on the baseline's plaque burden resulted in averages of 0.60 Accuracy and 0.23 Mathews Correlation Coefficient.

The prototype provided an XGBoost model capable of predicting plaque progression/regression changes in coronary vascular segments of patients treated with rosuvastatin therapy in 13 months. The prototype was the first system or method capable of addressing the problem of stratification of patients at risk of coronary plaque progression, using IVUS images and standard patient clinical data.

For the prototype, a predictive machine learning model was used to estimate the change in the percent of atheroma volume (ΔPAV) as the end-point, using baseline lumen and vessel contours from IVUS images and patient data (e.g., age, gender, body mass index, etc.). Predictions were performed at a region-wise (instead of frame-wise) level. The model developed to estimate ΔPAV for the prototype was based on an XGBoost regressor. Data was split into training/test groups, and a five-fold cross-validation was employed (at the patient level) with 20 repetitions to assess model performance. Model input included morphological features extracted from IVUS lumen and vessel contours. Note that the change in percent atheroma volume is the gold standard in terms of plaque quantification and assessments from the clinical point of view. The model was developed with a subset of the IBIS-4 data set, spanning 140 pullbacks from 81 patients, becoming the largest dataset ever used for plaque progression/regression prediction models.

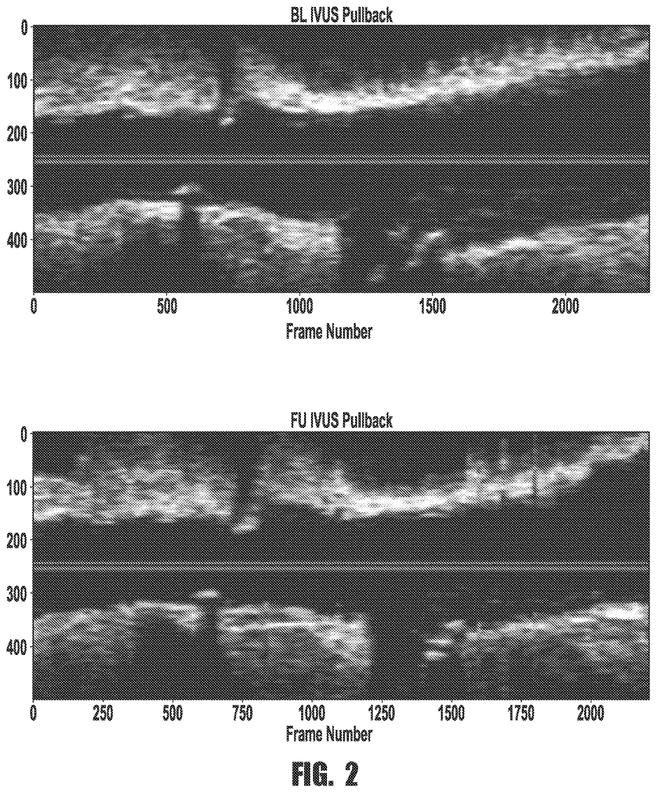

The IBIS-4 dataset (from which the dataset used for the prototype was obtained) was acquired as follows. A 20-MHz ultrasound catheter was used, at a speed of 0.5 mm/s. Frames for each pullback were manually selected between two anatomical landmarks (e.g., distal: side branch, proximal: Left main (LM) bifurcation or ostium of the right coronary artery (RCA)). Images were acquired at 30 frames per second, meaning that frame spacing is 1/60 mm. Baseline (BL) and Follow-up (FU) pullbacks were acquired 13 months apart. For each (BL, FU) pullback pair, the largest common region available was assessed using dedicated software, and many common matching frames within the pullbacks were identified. For the selected frames (mean frame spacing 0.4 mm), the lumen and the vessel contours were delineated using the same dedicated software (QIVUS). A total of 140 pullbacks from 81 patients were employed for the prototype. Referring to FIG. 2, illustrated is a (BL, FU) pullback pair of IVUS images (with the BL IVUS pullback shown at the top of FIG. 2 and the FU IVUS pullback shown at the bottom of FIG. 2), overlapped to area measurements and highlighting of regions with plaque progression or regression. Table 1, below, lists clinical variable definitions, demographics, and baseline characteristics for the 81 patient samples used for the model. For continuous variables the mean (std) is presented, and for Boolean variables the n (percentage of total) is presented.

TABLE 1
Baseline characteristics for the 81-patient sample
used for the prototype

| | |
|---|---|
| Age, AGE | 58.6 (9.9) |
| Male Sex, MSX (%) | 75 (93) |
| Body Mass Index, BMI | 27.5 (3.8) |
| Cholesterol | |
| High-density lipoproteins, HDL | 1.1 (0.3) |
| Low-density lipoproteins, LDL | 3.4 (0.9) |
| Cardiovascular risk factors (%) | |
| Diabetes, DIA | 8 (10) |
| Hypertension, HYT | 38 (47) |
| Hypercholesterolaemia, HYC | 35 (43) |
| Current Smoker, CSK | 34 (42) |
| Family history of CAD, FHC | 24 (31) |
| Renal failure, RFL | 3 (4) |
| Previous myocardial infarction, PMI | 2 (2) |
| Previous PCI, PPI | 1 (1) |
| Cardiovascular medications at 30 days (%) | |
| Statin use prior to enrolment (%), STA | 7 (9) |
| Aspirin, ASP | 81 (100) |
| Prasugrel, PRA | 63 (78) |
| Clopidogrel, CLO | 17 (21) |
| Any DAPT, DAPT | 80 (99) |
| Beta-blocker, BB | 77 (95) |
| ACE inhibitor, ACE | 60 (74) |

In Table 1, CAD stands for Coronary Artery Disease; PCI stands for Previous Percutaneous Infarction; DAPT stands for Dual Anti-Platelet Therapy; hypercholesterolaemia is defined based on total cholesterol >5.0 mmol or 190 mg/dL or requiring treatment; and renal failure is defined based on >60 eGFR. The original IBIS-4 study analyzed arterial cross-sectional geometry through area-based measurements and plaque composition. The later measurements were unavailable for the model. Table 2 lists the set of geometric descriptors the prototype model shared with the IBIS-4 study:

TABLE 2
Features used in the IBIS-4 study
Clinical frame-wise features

| | |
|---|---|
| Λ_{L} | Area enclosed by lumen contour, in mm². |
| Λ_{V} | Area enclosed by vessel (e.g., external |
| | elastic membrane (EEM)) |
| | contour, in mm². |
| Λ_{P} | Area between vessel and lumen |
| | contours, defined as the Λ_{V} − Λ_{L}, in |
| | mm². |
| PB | Plaque burden defined as (Λ_{P}/Λ_{V}) × 100, in %. |

Clinical condense features

| | |
|---|---|
| TLV | Volume of the lumen along a set of |
| | consecutive frames, integration of Λ_{L}, |
| | in mm³. |
| TVV | Volume of the vessel (e.g., EEM volume) |
| | along a set of consecutive |
| | frames, integration of Λ_{V}, in mm³. |
| TAV | Volume of the plaque along a set of |
| | consecutive frames, integration of |
| | Λ_{P}, in mm³. |
| PAV | Percentage atheroma volume |
| | (TAV/TVV) × 100, in %. |

Variable of interest

| | |
|---|---|
| ΔPAV | The difference of the PAV variable |
| | between follow-up (FU) and baseline |
| | (BL) at a specific region of interest, in %. |

The variable of interest for the model, also referred to as the target variable, is the difference in the percent atheroma volume (ΔPAV), follow-up (FU) minus baseline (BL), defined as in equation (1):

$$Δ P A V = P A {V}_{FU} - P A {V}_{BL} \qquad ( 1 )$$

where PAV_{FU} is the percent atheroma volume in the follow-up IVUS pullback and PAV_{BL} is the percent atheroma volume in the baseline IVUS pullback. Plaque progression is defined as ΔPAV>ε, regression is defined as ΔPAV<−ε, and −ε<ΔPAV<ε is regarded as stationary plaque for a given threshold ε. The model for which the prototype was developed used ε=0, as in the original IBIS-4 study, although various examples use other threshold values.

For any given frame of a pullback, BL or FU, for which the contour delineation of the lumen (L) and of the vessel (V) were available, a set of geometry-based descriptors were calculated, as shown in Table 3:

TABLE 3
proposed geometric features based on lumen
(L) and vessel (V) contours.

| | |
|---|---|
| ξ_{\|L\|V]} | Ratio between the maximum and |
| | minimum lumen (L) or vessel (V) |
| | diameters, measured using the line |
| | that runs through the image origin |
| | and joins opposite points through |
| | the corresponding contour. |
| ξ_{P} | Distance between the centroids |
| | of the lumen and vessel contours, |
| | divided by the average between the |
| | lumen minimum and maximum |
| | diameters. |
| ρ_{\|L\|V]} | Percentage of circumferential angle |
| | for which the ratio between plaque |
| | thickness and lumen (l) or vessel |
| | (v) radius is over 0.2. |
| [symbol] | Ratio between the maximum plaque |
| | thickness over the mean plaque thickness, |
| | along the circumferential direction. |
| ψ_{\|L\|V]} | Geometric definition of eccentricity |
| | for ellipses, \sqrt{1 − (min/max)²}, |
| | where min and max are the smallest |
| | and larger diameters of the lumen |
| | (L) or vessel (V). |
| φ_{\|L\|V]} | Geometric circularity of a polygon, |
| | 4πA/P², where A and P are the area |
| | and perimeter of the lumen (L) or |
| | vessel (V) contours. |
| τ_{\|L\|V]} | Curvature irregularity, defined as the |
| | difference between the maximum |
| | and minimum curvature of the lumen |
| | (L) or vessel (V) contour. |
| κ_{\|L\|V]} | Curvature roughness, reflects the |
| | lumen surface evenness concerning |
| | the curvature, smaller values |
| | representing a more circular or even |
| | surface, and a perfect circular lumen |
| | shape will have roughness of 1. It |
| | is calculated using the formula |
| | \sqrt{(r/2π)Σϰ²Δl} where r is the radius of |
| | the circle best fitting the lumen or |
| | vessel contour, ϰ is local curvature, |
| | and Δl is the local length between |
| | adjacent points. |

Frame-wise features were linearly interpolated to fill gaps between unevenly spaced frames and generate longitudinal signals. Features PB and Av were used to (manually) co-register BL and FU signals, resulting in anatomically consistent signals, with a uniform frame spacing of 60 frames/mm.

For each frame-wise feature (longitudinal signals) described in Tables 2 and 3, the first derivative was computed using a central finite difference approximation. As used herein, ∂f indicates the first derivative of the frame-wise feature f, and it is itself a frame-wise feature represented as a longitudinal signal.

A single IVUS pullback can be used to image a large portion of an epicardial coronary artery. Changes in the PB, from BL to FU, along the entire segment can either be positive (progression) or negative (regression), in a wide range of magnitudes. Furthermore, by considering a certain threshold ε, the outcome could be an unchanged PB. Since the target variable, ΔPAV, is defined over an arterial volume, regions of interest were defined within the pullback, for which the regression/classification model of the prototype analyzed and predicted the associated ΔPAV (e.g., value and/or sign).

Regions of interest (ROIs) were defined in 6 different ways for the model, to analyze the potential and limitations of the proposed classification methodology. Table 4 details the criteria used for ROI definition:

TABLE 4
Definition of criteria for the generation of ROIs.

| ROI Code | |
|---|---|
| Name | Criterion |
| FP | Full (complete) pullback. This |
| | criterion results in larger ROIs in |
| | which sub-regions of progression |
| | or regression may occur. |
| | Nevertheless, it is useful to establish |
| | a level of comparison and to |
| | separate sets for training and |
| | validation at a global level, as |
| | discussed herein. |
| ChPAV | Each pullback is divided into ROIs |
| | where the difference between |
| | BL and FU of PB is positive |
| | (or negative) in all (interpolated) |
| | frames of the ROI. Note that this |
| | approach for ROI definition is |
| | not applicable in clinical scenarios, |
| | where a new pullback is being |
| | analyzed and the FU data is |
| | not available. Nevertheless, for |
| | studying the potential of the method, |
| | this methodology provides an |
| | ideal ROI definition for APAV |
| | regression/classification models. |
| | Regions with \|PB_{FU} − PB_{BL} \| < 0.5% |
| | were discarded. Regions spanning |
| | less than 15 interpolated frames, i.e., |
| | less than 0.25 mm, were discarded. |
| PBR | The BL PB is binarized into three |
| | categories, PB ∈ [0, 30), PB ∈ |
| | [30, 60), and PB ∈ [60, 100]. Then, |
| | continuous frames with the |
| | same category are packed into |
| | ROIs. Regions spanning less than |
| | 15 interpolated frames, i.e., less |
| | than 0.25 mm, were discarded. |
| W30O10 | ROOs are divided using a fixed |
| | window size of 30 (interpolated) |
| | frames, which is shifted at 30 |
| | frames over the complete pullback. |
| | The ROI generation is repeated |
| | using 0, 10, 20 frames as offsets. |
| | Given the frames-spacing, these |
| | ROIs span 0.5 mm in length. |
| W60O20 | ROIs are divided using a fixed |
| | window size of 60 (interpolated) |
| | frames, which is shifted at 60 |
| | frames over the complete pullback. |
| | The ROI generation is repeated |
| | using 0, 20, 40 frames as offsets. |
| | Given the frames-spacing, these |
| | ROIs span 1.0 mm in length. |
| W120O20 | ROIs are divided using a fixed |
| | window size of 120 (interpolated) |
| | frames, which is shifted at 120 |
| | frames over the complete pullback. |
| | The ROI generation is repeated |
| | using 0, 20, 40, 60, 80, 100 |
| | frames as offsets. Given the |
| | frames-spacing, these ROIs span |
| | 2.0 mm in length. |

In various examples, ROIs are defined based on criteria such as those in Table 4, defined to have fixed length, or defined based on other features (e.g., lumen or EEM measurements, plaque burden measurements, etc., including features determined from frame-wise features such as signal processing features (e.g., via Fourier analysis), statistical features such as mean or standard deviation, differential features, etc. determined from plaque burden, etc. such as a first derivative of plaque burden, magnitude/phase of Fourier harmonics of plaque burden, etc.).

For each ROI, a set of features was generated using tools from statistics, signal processing, and information theory, shown in Table 5, below. Each of these ROI-wise features is a real number, and a set of n features represents each ROI as a point in an [symbol] space.

TABLE 5
List of condensed features defined within each ROI
Statistics over each frame-wise feature (ƒ)

| | |
|---|---|
| MED(ƒ) | The median of ƒ in the ROI. |
| IQR(ƒ) | The interquartile range of ƒ in the |
| | ROI, i.e., difference between |
| | percentiles 75%-25%. |
| ICR(ƒ) | The central 80% range of ƒ in the |
| | ROI, i.e., difference between |
| | percentiles 90%-10%. |

Information-theory over each frame-wise feature (ƒ)

| | |
|---|---|
| H(ƒ) | Shannon's Entropy from an approximation |
| | of the discrete probability |
| | function of the frame-wise feature ƒ. |

Fourier analysis over frame-wise feature (ƒ)

| | |
|---|---|
| FFT^{m} (ƒ) | The magnitude of the 1st Fourier |
| | harmonic, of the point-wise feature ƒ. |
| FFT^{p} (ƒ) | The phase of the 1st Fourier harmonic, |
| | of the point-wise feature ƒ. |

Macro features

| | |
|---|---|
| [symbol] | The arterial label, e.g., LAD, LCx, RCA, |
| | etc. that was interrogated. |
| [symbol] | The length of the ROI. |

A repeated stratified k-fold cross-validation (RSKFCV) strategy was used for training and testing. Folds were defined at the pullback level, i.e., using the FP criterion for ROI definition. Then, depending on the experiment, the corresponding ROIs for each pullback were accordingly assigned to the training or testing sets in the fold. Moreover, fixing the seed parameter of the random generator of the RSKFCV implementation ensured that all experiments are trained/tested over the same pullback sets, and what was changed were the ROIs defined inside the pullbacks. Stratification was based on the sign of the ΔPAV variable, at the pullback level.

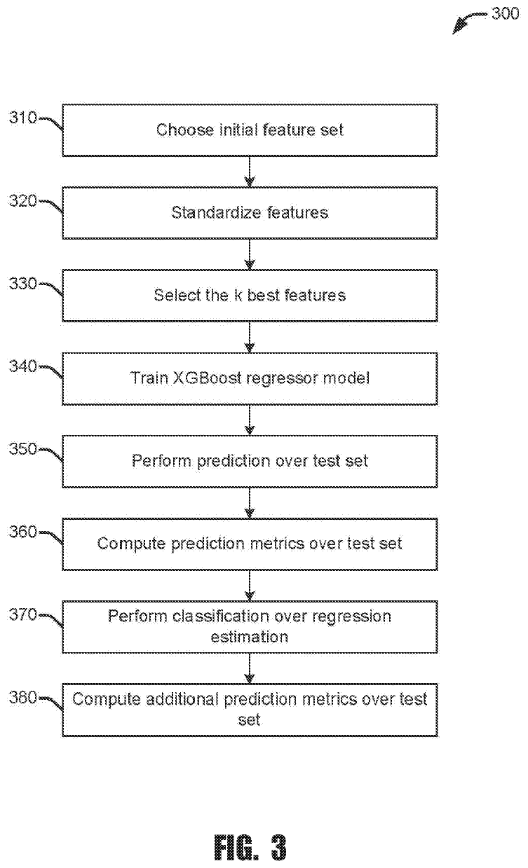

Referring to FIG. 3, illustrated is a method 300 of training and testing the prototype algorithm that was performed at each iteration. The prototype was implemented in Python v3.12, using the following packages: (a) numpy and pandas for management of tabular data; (b) scipy for implementation of statistics and information-based methods, as well as integration and interpolations; (c) scikit-learn for implementation of repeated stratified k-fold cross-validation, feature selection, and model performance metrics; (d) xgboost for the implementation of the XGBoost method; and (e) for computing Shapley additive explanations (SHAP) values.

At 310, the initial feature set was chosen. The following notation is used in connection with feature sets: (1) sa is the set of clinical features (e.g., from table 1), (2) [symbol] ^{b} is set [symbol] plus the set of geometric features from the original IBIS-4 study (e.g., from table 2), and (3) [symbol] is set [symbol] plus all of the condense features of table 5.

At 320, the features were standardized. The mean and standard deviation of the training set were computed. Standardization was as follows: \hat{d}=(d−d̅)/d̅, where d is each condensed feature from Tables 1 or 5, and d̅ and d̅ are the mean and standard deviation, respectively.

At 330, the k best features were selected using mutual information as a proxy classification function, as follows:
def mutual_information_proxy (X,y):
   return sklearn.feature_selection.mutual_info_classif(
      X,y, discrete_features=‘auto’, n_neighbors=9, proxy=mutual_information_proxy
nfeatures=32 # Or 8, depending on the test case.
proxy=mutual_information_proxy
featsel=sklearn.feature_selection.SelectKBest (proxy, k=nfeatures)
   Following the feature set notation discussed at 310, this resulted in the following feature sets: (1)

$${𝒮}_{k = 8}^{d} ,$$

which includes the best k=8 features of set s [symbol] and (2)

$${𝒮}_{k = 32}^{d} ,$$

which includes the best k=32 features of set [symbol] . In other examples, other numbers of features are used.

At 340, an XGBoost regressor model was trained (although other machine learning models can be employed in other example systems and methods). The same hyperparameter configuration was used in all experiments, which was defined empirically:
xgboostreg=xgboost.XGBRegressor(
   tree_method: ‘auto’, scale_pos_weight: 2.0, n_estimators: 256,
   max_depth: 12, learning_rate: 0.125, subsample: 0.95,
   colsample_bytree: 0.9, colsample_bylevel: 0.9, colsample_bynode: 1.0,
   objective: ‘reg: squarederror’, booster: ‘gbtree’, use_label_encoder: False, eval_metric: ‘mae’, random_state: 1, verbosity: 1, reg_lambda: 1, reg_alpha: 0)

At 350, prediction was performed over the test set, which involves standardization, feature selection (both using parameters from the training set), and then forward-passing through the model.

At 360, the following prediction metrics were computed over the test set: mean absolute error (MAE), mean square error (MSE), and Pearson's correlation coefficient (r).

At 370, classification was performed over the regression estimation using 0 as the threshold to classify in estimated progression or regression.

At 380, the following additional prediction metrics were computed over the test set: Accuracy (ACC), Mathews Correlation Coefficient (MCC), F1-score for ΔPAV<0 class (F1⁻), F1-score for ΔPAV >0 class (F1⁺), and the average F1-score between the two classes (F1^{a}).

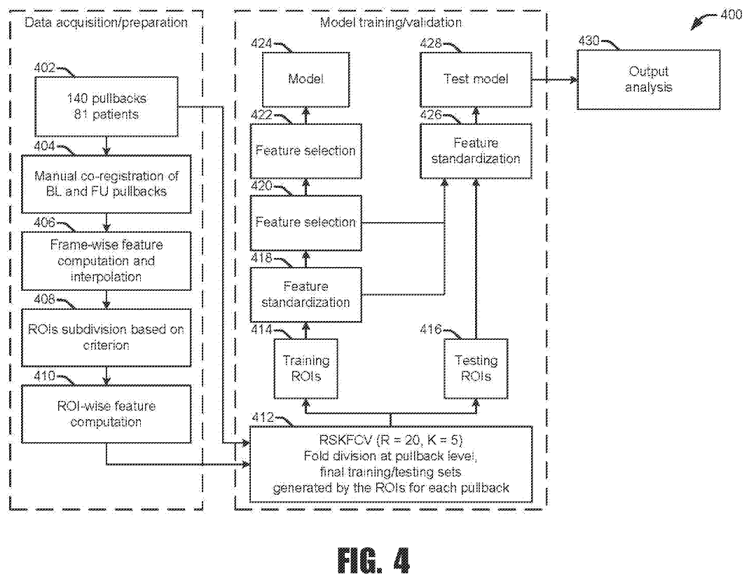

After iterations were completed, the mean and standard deviation of each prediction metric were gathered for assessing and comparing the performance. Referring to FIG. 4, illustrated is a flow diagram showing a flow diagram of performing one regression/classification experiment in connection with a prototype.

Data acquisition and preparation started at 402 with the dataset of 140 pullbacks from 81 patients. At 404, the BL and FU pullbacks were manually co-registered (in various examples automatic co-registration is employed based on maximizing or minimizing a similarity metric or function). At 406, signal interpolation and frame-wise feature computation are performed. At 408, the ROI is subdivided based on a selected criterion (e.g., from table 4, etc.). At 410, ROI-wise features are computed. At 412, cross-validation folds are defined at the pullback level, with actual partitions defined from corresponding ROIs, and divided into training ROIs and testing ROIs at 414 and 416, respectively.

All tests for the prototype were performed using the same data set partitions at pullback level, for the RSKFCV loop. Also, the XGBoost configuration remained fixed for all tests. Regarding the feature selection, it also remained fixed (whenever used), the only parameter that changed was the number of features to be selected (either 8 or 32).

Shapley additive explanations (SHAP) values were used to measure the impact of each feature on the output of the XGBoost model. The SHAP value of a feature was computed for each sample and then averaged over the test set. The larger the mean absolute SHAP value of a feature over the test set, the greater the impact on the prediction of the model relative to the mean prediction over the test set.

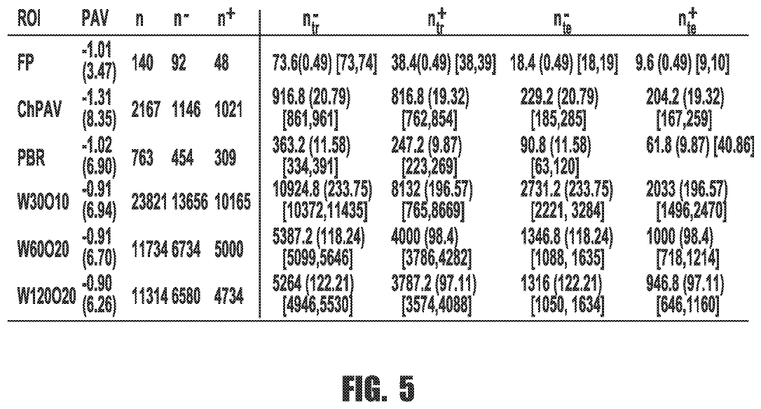

FIG. 5 is a table showing basic statistics for the ΔPAV variable, considering the different criteria to define the ROI partitions. Furthermore, the total number of samples per class (progression “+” or regression “−”) is reported, as well as the distribution in terms of mean, standard deviation (indicated via parentheses), min and max (indicated via brackets) of the number of samples per class for training (tr) and test (the) sets for the RSKFCV iterations. The class representing plaque progression (+) was the minority class for all partitions. Moreover, the class imbalance was more significant at FP partition (34%), and was most balanced for the ChPAV partition (47%), while the rest of the partitioning criteria exhibited a similar prevalence of the progression class (between 40% and 43%). In terms of sample size, the FP partition contained the smallest number of samples (n=140), followed by PBR with (n=763), which was almost 3 times smaller than the ChPAV partition sample size (n=2167). The other partitions were much larger in terms of sample size.

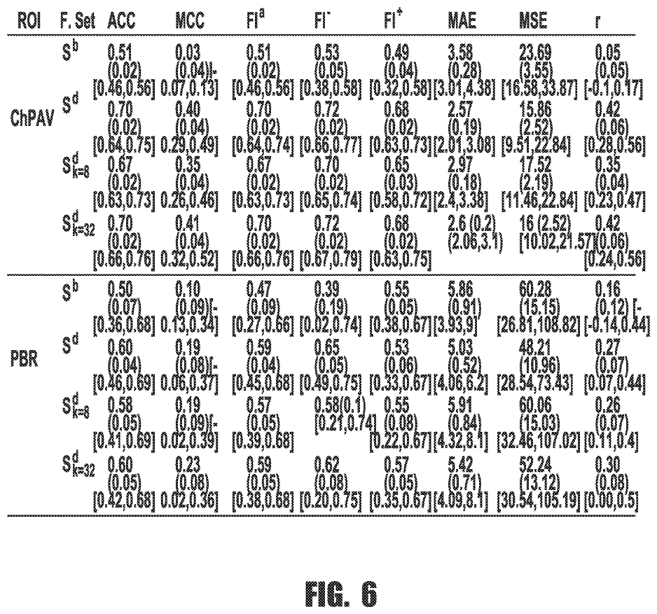

The statistics of the prediction metrics for all tests were determined. The tests were characterized by the dataset (ROI definition criterion), and by the set of features/feature-selection-strategy employed. For in-depth analysis, the ChPAV and PBR ROI definition criteria were considered. The ChPAV was useful as a reference because it represented the best possible ROI partition criterion since all frames in a given partition will render either progression or regression. In turn, the PBR criterion was chosen because it rendered the best results among the rest of the ROI partition criteria. FIG. 6 illustrates the statistics of the prediction metrics for the ChPAV and PBR ROI definition criteria as a result of training and testing with the same RSKFCV procedure, with 20 repetitions and 5 folds. Similar to FIG. 5, FIG. 6 shows the distribution in terms of mean, standard deviation (indicated via parentheses), min and max (indicated via brackets) of the number of samples across the RSKFCV procedure. The best-performing feature set according to the mean MCC metric was selected and used for an in-depth analysis, which was the

$${𝒮}_{k = 32}^{d}$$

feature set for both the ChPAV and PBR criteria.

The effects of the initial set of features, feature selection, and feature impact on model prediction were analyzed. The ChPAV and PBR scenarios analyzed used the same initial feature set and selection strategy,

$${𝒮}_{k = 32}^{d} .$$

The selection count and the selection ratio times the summation of the mean absolute SHAP value were analyzed. The mean absolute SHAP value of a feature was computed for each of the 100 models used during the RSKFCV procedure, using the corresponding test samples. Since feature selection of k=32 was performed prior to model training, the number of times a feature was selected throughout the 100 models was counted, the selection ratio was computed as the selection count over 100, and the selection ratio was used to weight the mean absolute SHAP value of the feature. Ordering by this weighted value and retrieving the top ten, the features with more impact on model predictions over the 100 models were obtained. From the initial set of 242 features, the selection process always selects the same 10 features (selection count equals 100), and this count rapidly dropped to around 20 for the 50th feature. From there, different features were rarely selected.

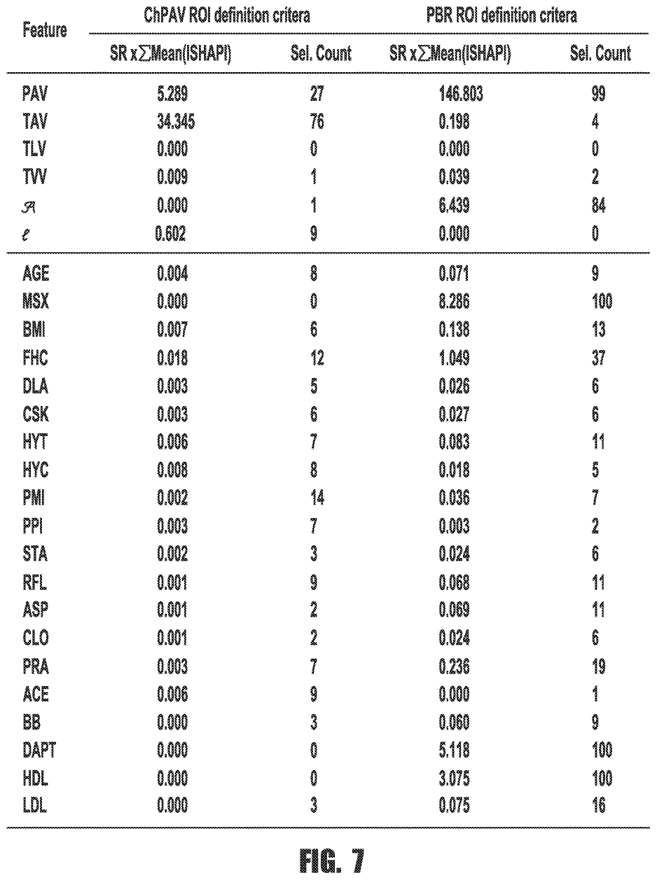

FIG. 7 presents the selection count and total mean absolute SHAP values times the selection ratio (SR) values for all clinical features defined in Tables 1 and 2, in addition to the arterial label [symbol] and ROI length [symbol] . As can be seen, the TLV, TVV, and & were rarely selected and when selected the impact in the models in terms of SHAP values was low, for the ChPAV and the PBR cases. The TAV impacted the ChPAV models considerably, but its contribution to the PBR models was poor. Overall, the PAV was the most impactful feature regarding the PBR models, while in the ChPAV case the PAV contributed weakly. Also, although the arterial label [symbol] reached a high selection count, its impact was weak on the PBR models and was close to zero on the ChPAV case. In terms of the clinical variables defined at the patient level, they were seldom selected within the ChPAV cases, and, consequently, the overall impact was close to zero. In turn, for the PBR case, the MSX, DAPT, and HDL were selected in all 100 models, although their overall global impact was weak. The rest of the clinical features were selected more often in the PBR than in the ChPAV case but displayed a poor relevance.

The models that resulted in the best prediction performance according to the MCC metric were identified. Regression plots and prediction metrics were determined for the ChPAV and PBR scenarios using

$${𝒮}_{k = 32}^{d}$$

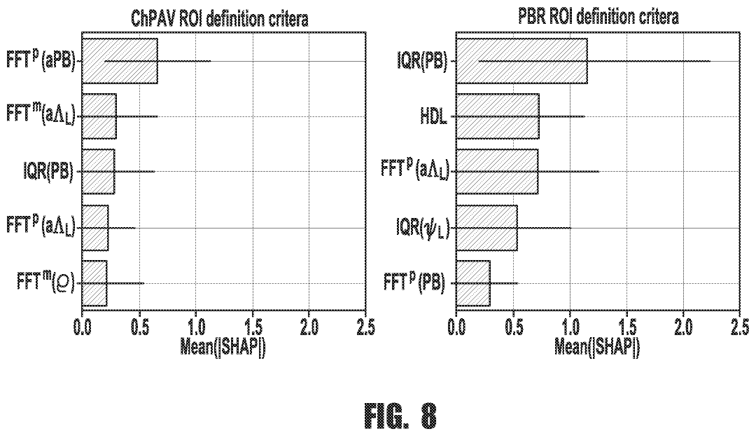

feature management setup. The ChPAV scenario renders better performance because ROIs are built, by definition, using the regression/progression feature as proxy. Using this scenario as a reference, the PBR case resulted in almost double the mean absolute error (MAE=5.04%), moderate linear, and Mathew's correlation (r=0.37 and MCC=0.36) which were 0.07 and 0.16 lower than the ChPAV scenario. In terms of accuracy and mean F1-scores, fairly good results (ACC=F1^{a}=0.65) were obtained for the PBR case, compared to 0.76 in the ChPAV case. FIG. 8 shows the mean absolute SHAP values of the five most influential features for the ChPAV and PBR cases. The top three features of each model were selected to visualize the entire ROI sample distributions in both scenarios. The foremost features were those related to plaque burden (PB) and lumen-area (Λ_{L}), according to mean absolute SHAP values, namely, the FFT^{p}(∂PB), FFT^{m}(∂)Λ_{L}, and IQR(PB) for the ChPAV case and the MED (PB) and FFT^{p}(∂)Λ_{L} for the PBR case. In the PBR case, the clinical feature HDL was the second most relevant feature for the model according to the mean absolute SHAP values. However, the mean HDL was not different between the regression/progression groups when taken as ROI level for ChPAV or PBR. This can be explained due to the fact that HDL is defined at the patient level, meaning that for all ROIs of a pullback (regardless of the ΔPAV sign), the HDL is the same. Therefore, it was the interaction of HDL with the other features that produced an impact on the model output. Analogous reasoning can be used to explain the impact of other patient-level clinical features on this model and the impact of other features for which no statistically significant difference in mean values among the regression/progression groups was observed.

The prototype employed an XGBoost regressor model to estimate the change in the percent of atheroma volume (ΔPAV) as the end-point, which is the standard measure of plaque evolution. Conventional models did not use such a machine learning model, nor used the ΔPAV, because they center on frame-wise prediction instead of region-wise prediction. The pre-processing and training methodology used 5-fold cross-validation (20-repetitions) at the patient level, which is considerably more robust than using cross-validation at the frame-level (as in conventional methods) since consecutive (and therefore very similar) frames are expected to provide highly correlated data into the training and validation phases. The input data for the prototype were vessel and lumen contours extracted from IVUS pullbacks, which are already the standard measurement in the clinic, in contrast to conventional methods that need VH-IVUS, OCT, or 3DQCA and segmentation of other structures such as lipids to obtain morphological features and to construct computational domains for complex computer simulations. Finally, the prototype model was developed with a subset of the IBIS-4 data set, spanning 140 pullbacks from 81 patients, becoming the largest data set ever used for plaque progression/regression prediction models, compared to state-of-the-art publications which used at most 9 pullbacks. Unlike conventional systems and methods, the prototype (1) employed a complete methodology to process contours extracted from IVUS to generate a comprehensive geometric description; (2) developed a machine-learning-based model to predict plaque progression/regression from such features and patient data. Thus, unlike conventional methods, the prototype employed a predictive model to estimate plaque evolution over time, based on information collected at baseline from IVUS images and clinical data.

When the full pullbacks were used for model training, that is, when the FP ROI definition criterion was employed, the models trained with feature sets containing geometric-based descriptors performed poorly. Such a result could be attributed to the fact that full pullbacks in the dataset cover different lengths and it is common for them to contain regions in which the plaque will increase (progression) and others in which the plaque decrease (regression). The compensation of these regions in the total ΔPAV is hard to predict when frame-wise features are condensed over the complete pullback. In other words, this confirms the fact that the plaque progression/regression problem is a local problem.

In turn, the FP ROI definition criterion was adequate to test the feature subset [symbol] , i.e., standalone clinical features defined at the patient level defined in Table 1. Under such a scenario, the method resulted in an average of 0.65 ACC and 0.21 MCC. It is worth noting that such predictive capabilities were similar to those obtained using the ROI definition criterion based on baseline plaque burden alone (PBR). Nonetheless, to correctly interpret such results, the following points should be taken into consideration: (1) The FP ROI criterion results in only 140 pullbacks from 81 patients, while the PBR ROI criterion resulted in 763 ROIs; (2) The feature set [symbol] was not used directly on the PBR ROI criterion tests because clinical features are defined at the patient level; and (3) Comparing FP and PBR criteria on all other feature subsets (including the [symbol] ) resulted in a clear superior performance of the PBR scenarios; and (4) Some clinical variables (see FIG. 7) impacted the predictions of the models based on the PBR ROI criterion.

The ChPAV ROI definition criterion was studied in connection with the prototype as a reference for comparison, and is not applicable in clinical scenarios, where a new pullback is analyzed by example systems and methods and the FU data is not available. However, the ChPAV ROI criterion highlights that the prototype reached outstanding prediction capabilities (averages of 0.70 ACC and 0.41 MCC) when the ROIs were homogeneous regarding regression or progression. In this scenario, the clinical features portrayed a weak influence on the model's output (see FIG. 7). In turn, the PBR ROI definition criterion resulted in averages of 0.60 ACC and 0.23 MCC for the case

$${𝒮}_{k = 32}^{d} ,$$

which were the best output among all ROI definition criteria that did not use follow up data (which would be unavailable in clinical use cases of example systems and methods).

The model that developed the prototype indicated that the locality of the plaque progression/regression problem can be tackled using strategies to divide each pullback into ROIs, where geometric features are retrieved using statistics-, information theory- and signal processing-based tools. The criteria to define these ROIs also produce an impact on model performance, because of the way in which the homogeneity, the sign, and the zeros of function PB_{FU}-PB_{BL} are characterized through each criterion.

Feature values are intrinsically related to the ROI definition criterion, such as the use of standalone clinical features in a full pullback analysis scenario. For example, clinical features had less predictive power when using the referential ChPAV criterion than when using the PBR criterion. This indicates that the predictive capabilities of these clinical features are moderate to poor (see FIG. 7). The explanation behind such behavior likely relies on the number of ROIs per pullback: the ChPAV case generates almost three times the number of ROIs than the PBR case. Since clinical variables are defined at the patient level, then it is expected that they lose predictive power when pullbacks contain regions where plaque either increases or diminishes.

Feature condensation worked better in the ChPAV scenario, which indicates some susceptibility of the condensation methods to the ROI definition criterion. It is possible that condensing a region with multiple progression/regression sub-regions undermines the predictive capabilities of the features.

Performing feature selection improved results. Other feature selection methods were tested, including sequential (forward/backward), Boruta, and conditional likelihood maximization, in addition to the SHAP-based feature selection, which provided the best results.

Additionally, dimensionality reduction techniques were tested to transform the high-dimensional space to a (more human-friendly) low-dimensional representation. Tested techniques included the use of Principal Component Analysis (PCA) and Uniform Manifold Approximation and Projection (UMAP).

Machine learning algorithms (e.g., the XGBoost algorithm employed by the prototype) often lack interpretability, making it challenging to understand the underlying features or the rationale behind their predictions. Using SHAP values or other feature selection techniques can highlight the feature impact on the model's prediction outcomes, providing clinicians with explanations for the decisions of example systems and methods, leading to an overall improvement in trust, acceptance, and adoption in clinical practice.

Although the prototype employed an XGBoost regressor, additional classification/regression models were studied, including: Nearest Neighbors, Support Vector Machines, Gaussian Process, Fully Connected Neural Networks, Gaussian Naive Bayes, Quadratic Discriminant Analysis, Gaussian Mixture Models, and Linear Regression. Overall, the XGBoost regressor delivered the best results regarding the metrics analyzed. In various examples, improved performance over the prototype can be obtained by (1) collecting more data and/or (2) envisioning a different analysis of ROIs. More data can be collected via accessing retrospective data of studies similar to IBIS4 and/or designing and executing new trials. ROIs can be analyzed in one of the following additional ways: (a) processing frame-wise features as spatial signals (in contrast to ROI-wise features strategies) using models for signal forecasting or natural language processing, such as Recurrent Neural Networks; (b) exploring alternative image-related features from the IVUS pullbacks as potential markers of plaque evolution, such as plaque composition (e.g., image texture, etc.), and localization (e.g., bifurcation, etc.); and (c) estimating hemodynamic and mechanical environments (through blood flow and structural computer simulations) to gather information on wall shear stresses or plaque inner stresses for use as additional or alternative features.

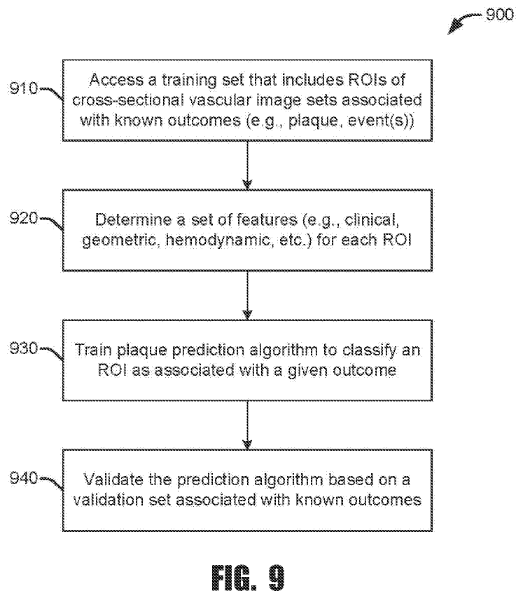

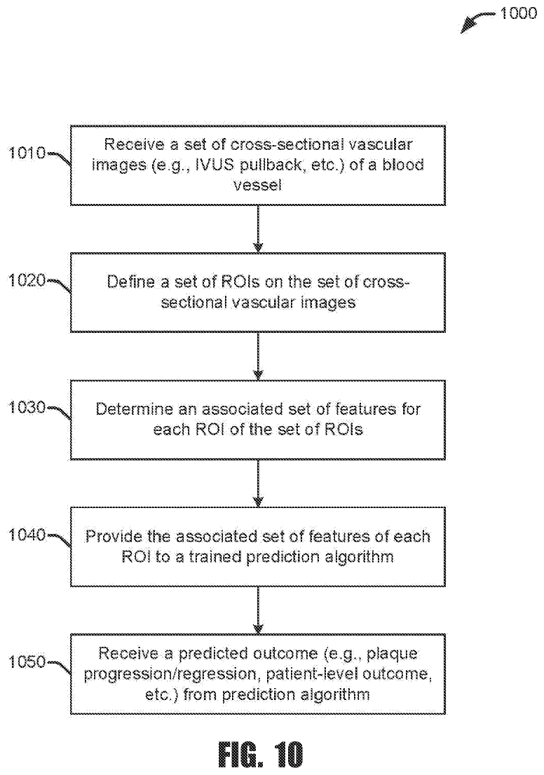

In view of the foregoing structural and functional features described above, example methods will be better appreciated with reference to FIGS. 9 and 10. While, for purposes of simplicity of explanation, the example methods of FIGS. 9 and 10 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement a method.

FIG. 9 illustrates a flowchart of an example method 900 for training and validating an outcome prediction algorithm to predict an outcome (e.g., plaque progression/regression in ROIs, a patient-level outcome such as whether or not a patient will have an adverse cardiovascular event, etc.) in response to a potential treatment from a set of cross-sectional vascular images. In other examples, the blocks of example method 900, are a set of machine-readable instructions on a non-transitory machine-readable medium or are a set of operations performed by a processing unit (e.g., of a outcome prediction training system comprising the processing unit and a memory storing the operations, etc.) executing machine-readable instructions as the operations.

At block 910, method 900 includes accessing a training set that includes ROIs from sets of cross-sectional vascular images (e.g., IVUS pullbacks, etc.), where each ROI of the training set has a known association (e.g., based on a follow-up set of cross-sectional vascular images) with an outcome (e.g., plaque progression/plaque regression or stationary plaque; a patient-level outcome; etc.).

At block 920, method 900 includes determining a set of features for each ROI. In various examples, the determined set of features includes features discussed herein (e.g., clinical, geometric, anatomic, hemodynamic, condensed, features derived therefrom, etc.). In various examples, the set of features includes features selected as more strongly predictive of potential outcome (e.g., plaque progression/regression, a patient-level outcome, etc.) than other potential features.

At block 930, method 900 includes training an outcome prediction algorithm to classify the ROI as associated with a predicted outcome (e.g., plaque progression/regression, a patient-level outcome, etc.). In various examples, the outcome prediction algorithm is a machine learning algorithm such as one of, or an ensemble of two or more of, a gradient-boosted decision tree algorithm (e.g., XGBoost), a Nearest Neighbors algorithm, a Support Vector Machine, a Gaussian Process, a Fully Connected Neural Network, a Gaussian Naive Bayes algorithm, a Quadratic Discriminant Analysis algorithm, a Gaussian Mixture Model, or a Linear Regression algorithm, employed for classification or regression.

In some examples, a gradient-boosted decision tree algorithm is employed as a classification algorithm (e.g., of plaque progression/regression, of a patient-level outcome, etc.). In other examples, a neural network-based model is employed, such as a bidirectional Long Short Term Memory (biLSTM) layer, followed by two dense layers working in parallel, where the first layer outputs the regression estimate (R-Dense), and the second layer outputs the classification (C-Dense) of the outcome (e.g., plaque progression/regression, a patient-level outcome, etc.). In one example, the biLSTM is configured with 200 hidden units with a hyperbolic tangent activation function which outputs a 200-length tensor to the Dense layers that have a single unit each, where the R-Dense layer has a linear activation function, and the C-Dense layer is configured with a Sigmoid activation function.

Various training methods are employed, depending on the example. In some examples, the Adam stochastic gradient descent optimizer method is employed in block 930.

At block 940, method 900 includes validating the outcome prediction algorithm based on a validation set with known associations with outcomes (e.g., plaque progression/regression or stationary plaque in some examples, patient-level outcomes, etc.). In some examples, subsets of a common dataset are used to select folds for training and cross-validation.

FIG. 10 illustrates a flowchart of an example method 1000 for employing an outcome prediction algorithm to predict an outcome (e.g., arterial plaque progression or regression, or in some examples stationary plaque; a patient-level outcome; etc.) will occur in response to a potential treatment, based on analysis of a set of ROIs of a set of cross-sectional vascular images (e.g., an IVUS pullback, etc.). In other examples, the blocks of example method 1000, are a set of machine-readable instructions on a non-transitory machine-readable medium or are a set of operations performed by a processing unit (e.g., of an outcome prediction system comprising the processing unit and a memory storing the operations, etc.) executing machine-readable instructions as the operations.

At block 1010, method 1000 includes accessing a set of cross-sectional vascular image frames of a blood vessel of a patient, which have segmented (e.g., manually or automatically) lumen and plaque.

At block 1020, method 1000 includes defining a set of ROIs on the set of cross-sectional vascular image frames, such as based on plaque burden or the first derivative of plaque burden, although other examples employ various other ROIs (fixed-length ROIs, ROIs defined based on lumen or EEM features, etc.).

At block 1030, method 1000 includes determining a set of features (e.g., features selected as more predictive of potential outcomes than other potential features) for each ROI of the set of ROIs.

At block 1040, method 1000 includes providing the set of features to a trained outcome prediction algorithm. In various examples, the outcome prediction algorithm is a machine learning algorithm such as a gradient-boosted decision tree algorithm (e.g., XGBoost), a Nearest Neighbors algorithm, a Support Vector Machine, a Gaussian Process, a Fully Connected Neural Network, a Gaussian Naive Bayes algorithm, a Quadratic Discriminant Analysis algorithm, a Gaussian Mixture Model, or a Linear Regression algorithm, employed for classification or regression.

At block 1050, method 1000 includes receiving a prediction indicating a predicted outcome (e.g., plaque progression/regression, a patient-level outcome, etc.) for the ROI from the trained outcome prediction algorithm.

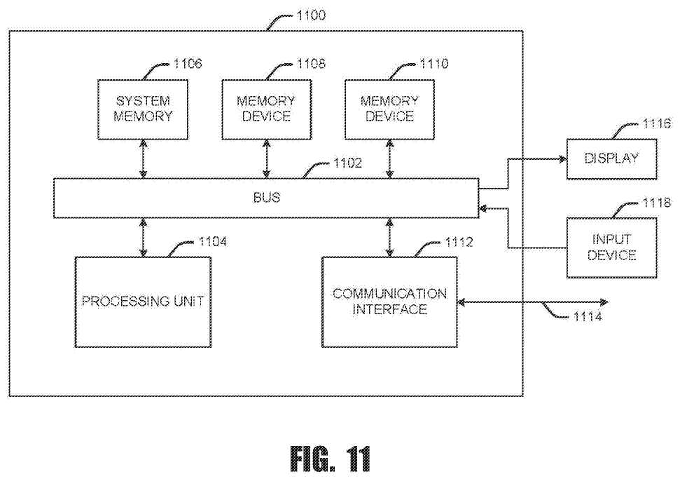

FIG. 11 is a schematic block diagram illustrating an exemplary system 1100 of hardware components capable of implementing examples of the systems and methods disclosed herein. The system 1100 can include various systems and subsystems. The system 1100 can be a personal computer, a laptop computer, a workstation, a computer system, an appliance, an application-specific integrated circuit (ASIC), a server, a server BladeCenter, a server farm, etc.

The system 1100 can include a system bus 1102, a processing unit 1104, a system memory 1106, memory devices 1108 and 1110, a communication interface 1112 (e.g., a network interface), a communication link 1114, a display 1116 (e.g., a video screen), and an input device 1118 (e.g., a keyboard, touch screen, and/or a mouse). The system bus 1102 can be in communication with the processing unit 1104 and the system memory 1106. The additional memory devices 1108 and 1110, such as a hard disk drive, server, standalone database, or other non-volatile memory, can also be in communication with the system bus 1102. The system bus 1102 interconnects the processing unit 1104, the memory devices 1106-1110, the communication interface 1112, the display 1116, and the input device 1118. In some examples, the system bus 1102 also interconnects an additional port (not shown), such as a universal serial bus (USB) port.

The processing unit 1104 can be a computing device and can include an application-specific integrated circuit (ASIC). The processing unit 1104 executes a set of instructions to implement the operations of examples disclosed herein. The processing unit can include a processing core.

The additional memory devices 1106, 1108, and 1110 can store data, programs, instructions, database queries in text or compiled form, and any other information that may be needed to operate a computer. The memories 1106, 1108 and 1110 can be implemented as computer-readable media (integrated or removable), such as a memory card, disk drive, compact disk (CD), or server accessible over a network. In certain examples, the memories 1106, 1108 and 1110 can comprise text, images, video, and/or audio, portions of which can be available in formats comprehensible to human beings. Additionally or alternatively, the system 1100 can access an external data source or query source through the communication interface 1112, which can communicate with the system bus 1102 and the communication link 1114.

In operation, the system 1100 can be used to implement one or more parts of a system in accordance with the present invention. Computer executable logic for implementing the diagnostic system resides on one or more of the system memory 1106, and the memory devices 1108 and 1110 in accordance with certain examples. The processing unit 1104 executes one or more computer executable instructions originating from the system memory 1106 and the memory devices 1108 and 1110. The term “computer readable medium” as used herein refers to a medium that participates in providing instructions to the processing unit 1104 for execution. This medium may be distributed across multiple discrete assemblies all operatively connected to a common processor or set of related processors.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on.

1. A machine-readable medium having machine executable instructions that cause a processor core to execute operations, the operations comprising:
accessing a set of segmented cross-sectional image frames of a blood vessel of a patient;
defining a set of regions of interest (ROIs) of the set of segmented cross-sectional image frames;
determining a set of features for a ROI of the set of ROIs;
providing the set of features to a trained outcome prediction algorithm; and
receiving a prediction for the ROI from the trained outcome prediction algorithm, wherein the prediction indicates one of a patient-level outcome, plaque progression, plaque regression, or stationary plaque.

2. The machine-readable medium of claim 1, wherein the prediction indicates one of the plaque progression or the plaque regression.

3. The machine-readable medium of claim 1, wherein the trained outcome prediction algorithm is a machine learning algorithm, wherein the machine learning algorithm is one of a classification algorithm or a regression algorithm.

4. The machine-readable medium of claim 3, wherein the machine learning algorithm comprises one of a gradient-boosted decision tree algorithm, a Nearest Neighbors algorithm, a Support Vector Machine, a Gaussian Process, a Fully Connected Neural Network, a Gaussian Naive Bayes algorithm, a Quadratic Discriminant Analysis algorithm, a Gaussian Mixture Model, or a Linear Regression algorithm.

5. The machine-readable medium of claim 1, wherein the set of ROIs are defined based on one of: a fixed length, a set of plaque burden values determined for the set of segmented cross-sectional image frames, a feature derived from the set of plaque burden values, a set of lumen measurements determined for the set of segmented cross-sectional image frames, a feature derived from the set of lumen measurements, a set of external elastic membrane (EEM) measurements determined for the set of segmented cross-sectional image frames, or a feature derived from the set of EEM measurements.

6. The machine-readable medium of claim 1, wherein the set of features comprises a clinical feature associated with the patient or a feature derived from the clinical feature.

7. The machine-readable medium of claim 6, wherein the clinical feature is a measure of high-density lipoprotein.

8. The machine-readable medium of claim 1, wherein the set of features comprises a condensed feature defined over the ROI.

9. The machine-readable medium of claim 8, wherein the condensed feature is one of a statistical measure of a frame-wise feature or a signal processing measure of a frame-wise feature.

10. The machine-readable medium of claim 9, wherein the frame-wise feature is one of an area enclosed by a lumen contour or a plaque burden.

11. A system, comprising:
a memory for storing machine-readable instructions; and
a processor core for accessing the machine-readable instructions and executing the machine-readable instructions as operations, the operations comprising:
accessing a set of segmented cross-sectional image frames of a blood vessel of a patient;
defining a set of regions of interest (ROIs) of the set of segmented cross-sectional image frames;
determining a set of features for a ROI of the set of ROIs;
providing the set of features to a trained outcome prediction algorithm; and
receiving a prediction for the ROI from the trained outcome prediction algorithm, wherein the prediction indicates one of a patient-level outcome, plaque progression, plaque regression, or stationary plaque.

12. The system of claim 11, wherein the trained outcome prediction algorithm is a machine learning algorithm, wherein the machine learning algorithm is one of a classification algorithm or a regression algorithm.

13. The system of claim 11, wherein the set of ROIs are defined based on one of: a fixed length, a set of plaque burden values determined for the set of segmented cross-sectional image frames, a feature derived from the set of plaque burden values, a set of lumen measurements determined for the set of segmented cross-sectional image frames, a feature derived from the set of lumen measurements, a set of external elastic membrane (EEM) measurements determined for the set of segmented cross-sectional image frames, or a feature derived from the set of EEM measurements.

14. The system of claim 11, wherein the set of features comprises a measure of high-density lipoprotein associated with the patient.

15. The system of claim 11, wherein the set of features comprises a condensed feature defined over the ROI, the condensed feature is one of a statistical measure of a frame-wise feature or a signal processing measure of a frame-wise feature, and the frame-wise feature is one of an area enclosed by a lumen contour or a plaque burden.

16. A method, comprising:
accessing a set of segmented cross-sectional image frames of a blood vessel of a patient;
defining a set of regions of interest (ROIs) of the set of segmented cross-sectional image frames;
determining a set of features for a ROI of the set of ROIs;
providing the set of features to a trained outcome prediction algorithm; and
receiving a prediction for the ROI from the trained outcome prediction algorithm, wherein the prediction indicates one of a patient-level outcome, plaque progression, plaque regression, or stationary plaque.

17. The method of claim 16, wherein the trained outcome prediction algorithm is a machine learning algorithm, wherein the machine learning algorithm is one of a classification algorithm or a regression algorithm.

18. The method of claim 16, wherein the set of ROIs are defined based on one of: a fixed length, a set of plaque burden values determined for the set of segmented cross-sectional image frames, a feature derived from the set of plaque burden values, a set of lumen measurements determined for the set of segmented cross-sectional image frames, a feature derived from the set of lumen measurements, a set of external elastic membrane (EEM) measurements determined for the set of segmented cross-sectional image frames, or a feature derived from the set of EEM measurements.

19. The method of claim 16, wherein the set of features comprises a measure of high-density lipoprotein associated with the patient.

20. The method of claim 16, wherein the set of features comprises a condensed feature defined over the ROI, the condensed feature is one of a statistical measure of a frame-wise feature or a signal processing measure of a frame-wise feature, and the frame-wise feature is one of an area enclosed by a lumen contour or a plaque burden.
